(19) United States
(12) Patent Application Publication
(10) Pub. No.: US 2008/0188892 A1
(43) Pub. Date: Aug. 7, 2008

(54) VASCULAR OCCLUSION DEVICE

(75) Inventors: BRIAN L. BATES, Bloomington, IN (US); Carlos E. Ruiz, New York, NY (US); Christopher L. Hruska, Indianapolis, IN (US); Kurt J. Tekulve, Ellettsville, IN (US); Ram H. Paul, Bloomington, IN (US)

Correspondence Address: BRINKS HOFER GILSON & LIONE/CHICAGO/COOK, PO BOX 10395, CHICAGO, IL, 60610, US

(73) Assignee: COOK INCORPORATED, Bloomington, IN (US)

(21) Appl. No.: 12/022,693

(22) Filed: Jan. 30, 2008

Related U.S. Application Data
(60) Provisional application No. 60/898,921, filed on Feb. 1, 2007.

(51) Int. Cl. A61B 17/08

(52) U.S. Cl. 606/213

(57) ABSTRACT

A vascular occlusion device for occluding a body cavity. The device includes an elongate flexible member having a proximal portion extending to a distal portion and a radially compressible substance being disposed therebetween. The substance is configured to promote growth of body tissue and may include an extracellular matrix such as small intestine submucosa. The proximal and distal end portions are anchored to the cavity walls such that the position of the substance within the body cavity promotes the growth of body tissue to occlude the body cavity. In one example, the body cavity includes a patent foramen ovale.

### CROSS-REFERENCE TO RELATED APPLICATIONS

This application claims the benefit of U.S. Provisional Application Ser. No. 60/898,921, filed on Feb. 1, 2007, entitled “VASCULAR OCCLUSION DEVICE,” the entire contents of which are incorporated herein by reference.

### BACKGROUND OF THE INVENTION

1. Field of the Invention

The present invention generally relates to vascular occlusion devices. More specifically, the invention relates to a vascular occlusion device for repairing an atrial septal defect.

2. Description of Related Art

A number of different devices may be used to occlude a body cavity, for example, a blood vessel. When it is desirable to quickly occlude a blood vessel, an inflatable balloon may be used. However, balloon's have the disadvantage of being temporary. Another example of an occlusion device includes embolization coils. Embolization coils are permanent and promote blood clots or tissue growth over a period of time, thereby occluding the body cavity. However, while the blood clots or the tissue grows, blood may continue to flow past the coil and through the body cavity. It may take a significant period of time for sufficient tissue to grow to fully occlude the body cavity. This leaves a patient open to a risk of injury from the condition which requires the body cavity be occluded.

In view of the above, it is apparent that there exists a need for an improved vascular occlusion device.

### SUMMARY OF THE INVENTION

In satisfying the above need, as well as overcoming the enumerated drawbacks and other limitations of the related art, the present invention provides a vascular occlusion device for occluding a body cavity. The device comprises an elongate flexible member including a proximal portion extending to a distal portion and a radially compressible substance disposed between the proximal portion and the distal portion. The substance comprises an extra cellular matrix and is configured to promote body tissue growth within the body cavity to occlude the body cavity.

In some embodiments, the elongate flexible member is a coil. In others, the proximal and distal portions of the elongate flexible member are curled into loops, and the loops may optionally be curled about axes substantially perpendicular to the elongate flexible member. In yet other examples the proximal portion may include a threaded end.

In other embodiments, the substance includes an extracellular matrix. The extracellular matrix may further include small intestine submucosa (SIS). In some examples, the SIS is compressed for passage through a lumen of a sheath and is expanded when disposed outside of the lumen.

In still other embodiments, the elongate flexible member is made of a shape memory material. The shape memory material includes various nickel-titanium alloys, known more commonly as Nitinol.

Still other embodiments of the present invention include a vascular occlusion assembly for occluding a body cavity. The assembly comprises a delivery apparatus including an outer sheath having a proximal end extending to a distal end and defining a lumen therein. An inner catheter is disposed within the lumen and has a proximal segment extending to a distal segment. The outer sheath is configured to translate axially relative to the inner catheter.

The assembly also includes one of the vascular occlusion devices described above having an elongate flexible member disposed within the lumen and releasably coupled to the distal segment of the inner catheter. The elongate flexible member has a proximal portion extending to a distal portion and an extracellular matrix disposed therebetween. The extracellular matrix is radially compressible for passage through the lumen and configured to expand and promote body tissue growth when disposed within the body cavity. The elongate flexible member is coaxially arranged within the lumen such that the extracellular matrix is compressed within the lumen. The occlusion device is deployable through the distal end of the outer sheath by relative axial movement of the outer sheath and the extracellular matrix is expanded after deployment.

In another embodiment, the proximal portion includes a threaded end. The threaded end couples the proximal portion of the elongate flexible member to the distal segment of the inner catheter.

The present invention also includes a method of occluding a body cavity. The method comprises conveying an occlusion device having an elongate flexible member including a compressed extracellular matrix to the body cavity by means of a delivery apparatus; positioning the elongate flexible member within the body cavity; expanding the extracellular matrix within the body cavity; coupling a proximal portion and a distal portion of the elongate flexible member to walls of the body cavity; detaching the elongate flexible member from an inner catheter of the delivery apparatus; and promoting tissue growth to occlude the body cavity. In some embodiments, the body cavity includes a patent foramen ovale in a heart.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 is a side view of a straight device for occluding a body cavity;

FIG. 2a is a side view of a curled device for occluding a body cavity;

FIG. 2b is a top view of the curled device of FIG. 2;

FIG. 3a is a plan view of a catheter assembly for introducing the device of FIG. 1 or FIG. 2 into the body cavity;

FIG. 3b is a plan view of the components of the assembly of FIG. 3a;

FIG. 4a is a section view of a human heart showing the assembly of FIG. 3a introducing the device of FIG. 2 into a patent foramen ovale;

FIG. 4b is a section view showing the device of FIG. 2 in position within the patent foramen ovale; and

FIG. 5 is a flow chart illustrating a method of occluding a body cavity.

### DETAILED DESCRIPTION

Referring now to FIG. 1, a device embodying the principles of the present invention is illustrated therein and designated at 10. As its primary components, the device 10 includes an elongate flexible member 12 including a proximal portion 16 extending to a distal portion 18. A radially compressible substance 14 is disposed between the proximal and distal portions 16 and 18. In some embodiments, the proximal portion 16 may include a threaded end 20.

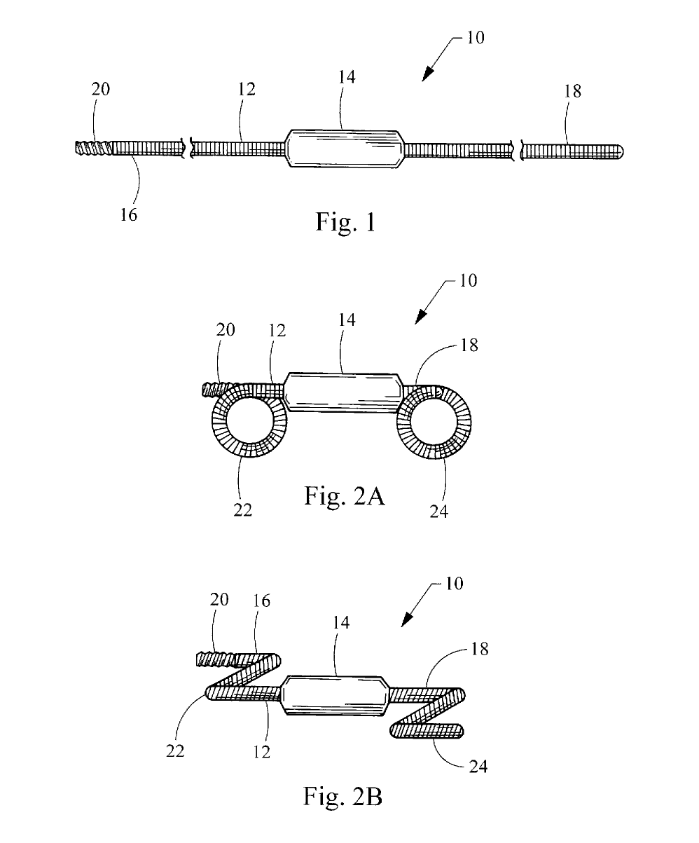

In one embodiment, the elongate flexible member 12 is formed as a longitudinal coil. The longitudinal coil may be substantially straight along an axial length of the device 10 as shown in FIG. 1. On the other hand, as shown in FIGS. 2a and 2b, the proximal and distal portions 16 and 18 of the elongate flexible member 12 may respectively include at least one proximal loop 22 and at least one distal loop 24. Additionally, the proximal and distal loops 22 and 24 may be curled about axes substantially perpendicular to the axial length of the elongate flexible member 12. Depending on the needs of a particular application, any number of loops may be provided and the loops may have any appropriate orientations with respect to the elongate flexible member 12.

The substance 14 may be any suitable compressible material for promoting tissue growth within a body cavity. In one embodiment, the substance 14 is made of connective tissue material, for example, extracellular matrix (ECM). As known, ECM is a complex structural entity surrounding and supporting cells found within tissues. More specifically, ECM includes structural proteins (for example, collagen and elastin), specialized protein (for example, fibrillin, fibronectin, and laminin), and proteoglycans, a protein core to which are attached long chains of repeating disaccharide units termed glycosaminoglycans.

In a preferred embodiment, the extracellular matrix is comprised of small intestinal submucosa (SIS). As known, SIS is a resorbable, acellular, naturally occurring tissue matrix composed of ECM proteins and various growth factors. SIS is derived from the porcine jejunum and functions as a remodeling bioscaffold for tissue repair. SIS has characteristics of an ideal tissue engineered biomaterial and can act as a bioscaffold for remodeling of many body tissues including skin, body wall, musculoskeletal structure, urinary bladder, and also supports new blood vessel growth. SIS may be used to induce site-specific remodeling of both organs and tissues depending on the site of implantation. In practice, host cells are stimulated to proliferate and differentiate into site-specific connective tissue structures, which have been shown to completely replace the SIS material in time.

In this embodiment, SIS is used to adhere to walls of a body cavity in which the device 10 is deployed and to promote body tissue growth within the body cavity. SIS has a natural adherence or wetability to body fluids and connective cells comprising the connective tissue of the walls of a body cavity. Since the device 10 is intended to permanently occlude the body cavity, the device 10 is positioned such that host cells of the wall will adhere to the SIS and subsequently differentiate, growing into the SIS and eventually occluding the body cavity with the tissue of the walls to which the substance 14 was originally adhered.

At least part of the elongate flexible member 12 of the device 10 may be made of any suitable material, for example, a superelastic material, stainless steel wire, cobalt-chromium-nickel-molybdenum-iron alloy, cobalt-chrome alloy, or stress relieved metal (e.g. platinum). It is understood that the elongate flexible member 12 may preferably be formed of any suitable material that will result in a device 10 capable of being percutaneously inserted and deployed within a body cavity, such as shape memory material. Shape memory materials or alloys have the desirable property of becoming rigid, i.e., returning to a remembered state, when heated above a transition temperature. A shape memory alloy suitable for the present invention is Ni—Ti available under the more commonly known name Nitinol. When this material is heated above the transition temperature, the material undergoes a phase transformation from martensite to austenic, such that material returns to its remembered state. The transition temperature is dependent on the relative proportions of the alloying elements Ni and Ti and the optional inclusion of alloying additives.

In one embodiment, the elongate flexible member 12 of the device 10 is made of magnetic resonance imaging (MRI) compatible material, including materials such as a polypropylene, nitinol, titanium, copper, or other metals that do not disturb MRI images adversely. The elongate flexible member 12 of the device 10 may also be made of radiopaque material, including tantalum, barium sulfate, tungsten carbide, bismuth oxide, barium sulfate, platinum or alloys thereof, and cobalt alloys.

In one embodiment, the elongate flexible member 12 is made from Nitinol with a transition temperature that is slightly below a normal body temperature of humans, which is about 98.6° F. Thus, when the device 10 is deployed in a body vessel and exposed to normal body temperature, the alloy of the device 10 will transform to austenite, that is the remembered state. For the embodiment of FIGS. 2a and 2b the remembered state includes the proximal and distal loops 22 and 24 when the device 10 is deployed in the body cavity. If it is ever necessary to remove the device 10 from the body cavity, the device 10 is cooled to transform the material to martensite which is more ductile than austenite, making the device 10 more malleable. As a result, the device 10 can be more easily collapsed and pulled into a lumen of a catheter for removal.

In another embodiment, the device 10 is made from Nitinol with a transition temperature that is above normal body temperature of humans, which is about 98.6° F. Thus, when the device 10 is deployed in a body vessel and exposed to normal body temperature, the device 10 is in the martensitic state so that the device 10 is sufficiently ductile to bend or form into a desired shape. For the embodiment of FIGS. 2a and 2b this is the state including the proximal and distal loops 22 and 24. In the event it ever becomes necessary to remove the device 10, the device 10 is heated to transform the alloy to austenite so that the device 10 becomes rigid and returns to a remembered state, which for the device 10 is a substantially straight state such as that shown in FIG. 1.

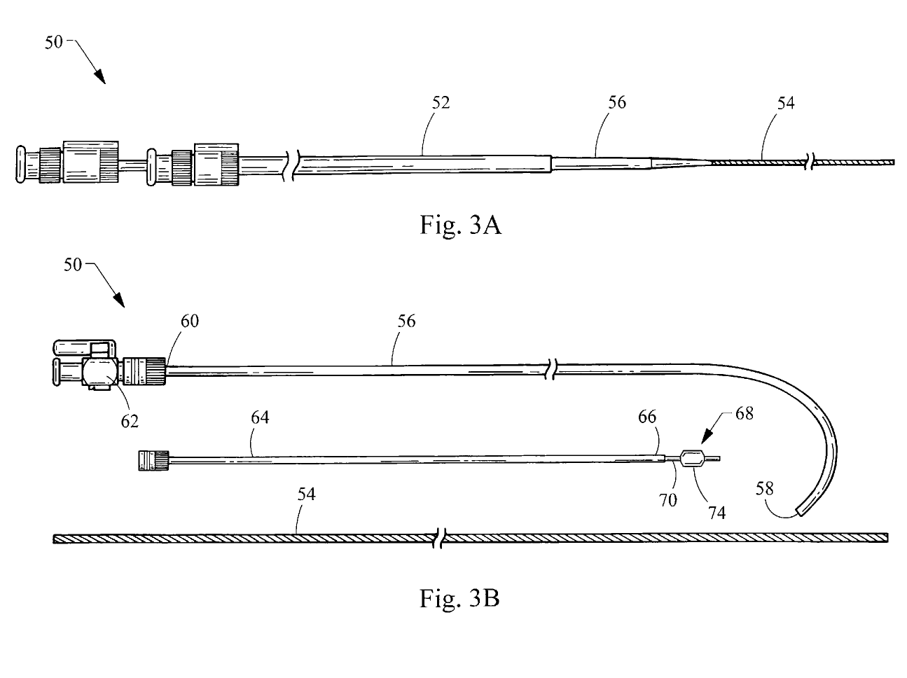

FIGS. 3a and 3b depict a delivery assembly 50 for introducing and retrieving a device 68 for occluding a body cavity in accordance with another embodiment of the present invention. As shown, the delivery assembly 50 includes a polytetrafluoroethylene (PTFE) introducer sheath 52 for percutaneously introducing an outer sheath 56 into a body vessel. Of course, any other suitable material for the introducer sheath 52 may be used without falling beyond the scope or spirit of the present invention. The introducer sheath 52 may have any suitable size, for example, between about three-french to eight-french. The introducer sheath 52 serves to allow the outer sheath 56 and an inner catheter 64 to be percutaneously inserted to a desired location in a body cavity through the body vessel. It should be understood that the inner catheter 64 is not limited to catheters, but may include any elongate pushing member, for example, a stylet. The introducer sheath 52 receives the outer sheath 56 and provides stability to the outer sheath 56 at a desired entry location of the body vessel. For example, the introducer sheath 52 is held stationary within a common visceral artery, and adds stability to the outer sheath 56, as the outer sheath 56 is advanced through the introducer sheath 52 to an occlusion area in the body cavity.

As shown, the assembly 50 may also include a wire guide 54 configured to be percutaneously inserted within the body vessel to guide the outer sheath 56 to the occlusion area. The wire guide 54 provides the outer sheath 56 with a path to follow as it is advanced within the body vessel. The size of the wire guide 54 is based on the inside diameter of the outer sheath 56 and the diameter of the body vessels that must be traversed to reach the desired body cavity.

When a distal end 58 of the outer sheath 56 is at the desired location in the body cavity, the wire guide 54 is removed and the occlusion device 68, having a proximal portion 70 releasably coupled by, for example, a threaded end attached to a distal segment 66 of the inner catheter 64, is inserted into the outer sheath 56. While one example uses the threaded end for coupling the occlusion device 66 to the inner catheter 64, other examples may use any other appropriate coupling means including, but not limited to, hooks, latches, or other devices. The inner catheter 64 is advanced through the outer sheath 56 for deployment of the occlusion device 68 through the distal end 58 to occlude, for example, a patent foramen ovale in a human heart.

As shown, the outer sheath 56 also has a proximal end 60 and a hub 62 to receive the occlusion device 68 and the inner catheter 64 to be advanced therethrough. When the occlusion device 68 is inside of the outer sheath 56 the occlusion device 68 takes a radially compressed form. The size of the outer sheath 56 is based on the size of the body vessel in which it percutaneously inserts, and the size of the occlusion device 68.

In the present embodiment, the occlusion device 68 and inner catheter 64 are coaxially disposed through the outer sheath 56, following removal of the wire guide 54, in order to position the occlusion device 68 to occlude, for example, the patent foramen ovale. The occlusion device 68 is guided through the outer sheath 56 by the inner catheter 64, preferably from the hub 62, and exits from the distal end 58 of the outer sheath 56 at a location within the heart where occlusion of the patent foramen oval is desired.

Likewise, this embodiment may also retrieve the occlusion device 68, should it ever become necessary. Retrieval may be accomplished by positioning the distal end 58 of the outer sheath 56 adjacent the deployed occlusion device 68 in the body cavity. The inner catheter 64 is advanced through the outer sheath 56 until the distal segment 66 protrudes from the distal end 58 of the outer sheath 56. The distal segment 66 is coupled to the proximal portion 70 of the occlusion device 68. After the occlusion device 68 has been freed from walls of the body cavity, the inner catheter 64 is retracted proximally, drawing the occlusion device 68 into the outer sheath 56.

It is understood that the assembly described above is merely one example of an assembly that may be used to deploy the device in a body vessel. Of course, other apparatus, assemblies and systems may be used to deploy any embodiment of the device without falling beyond the scope or spirit of the present invention.

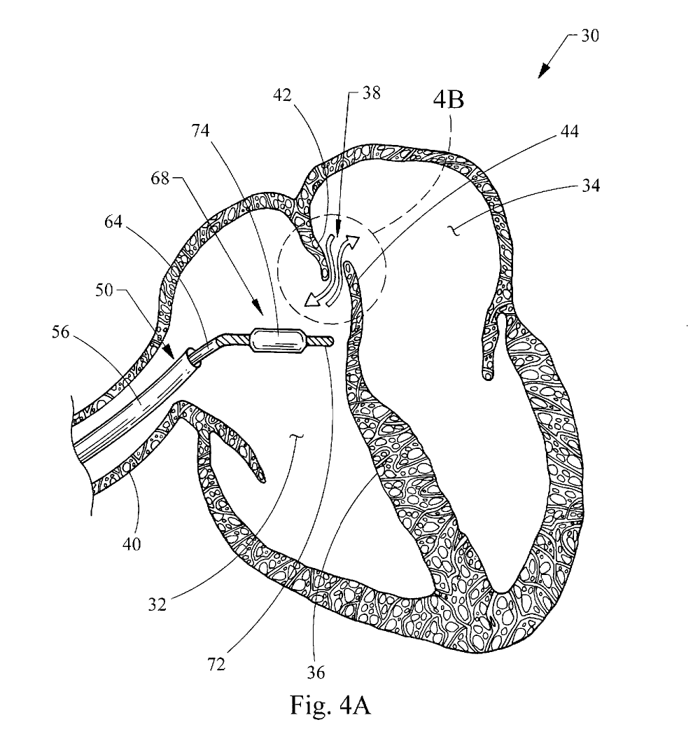

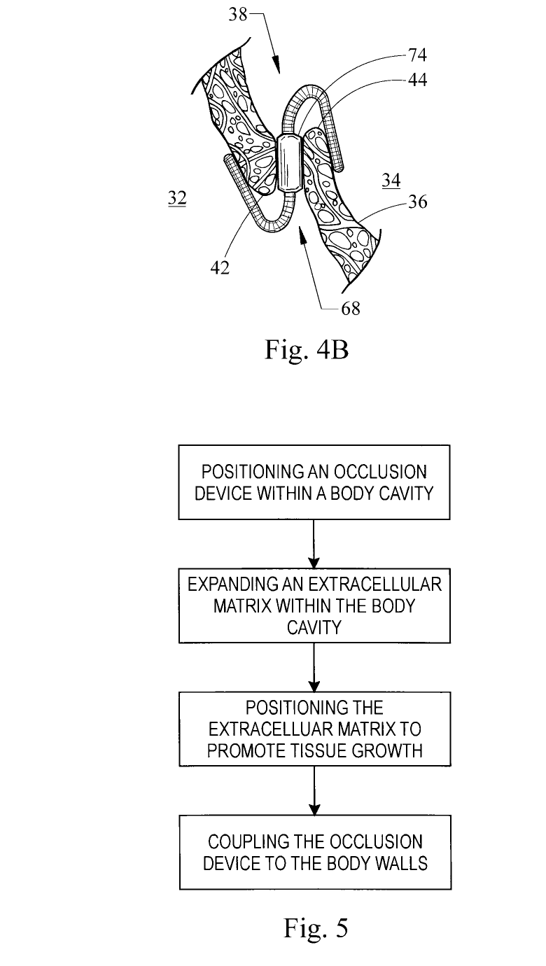

As mentioned above, one exemplary application of the delivery assembly 50 may be to treat a patent foramen ovale in a human heart 30 as shown in FIGS. 4a and 4b. It should be noted that this is merely one example and the delivery assembly 50 may be used in a variety of other applications to occlude various other body cavities without departing from the scope or spirit of the present invention. FIG. 4a shows a sectional view of a human heart 30 having a right atrium 32 and a left atrium 34. An atrial septum 36 divides the right atrium 32 from the left atrium 34 and includes a patent foramen oval 38. The patent foramen oval 38 is an opening in the atrial septum 36 that allows blood in the right and left atria 32 and 34 to fluidly communicate therebetween.

In a fetus, a foramen ovale is a natural hole in the atrial septum 38 that allows blood to bypass the fetus' lungs when in a mother's womb since the fetus relies on the mother to provide oxygen through the umbilical cord. At birth the foramen ovale normally closes when increased blood pressure in the left atrium forces the opening to close. Overt time tissue growth closes the opening permanently. However, in some people the opening does not close permanently, in which case the opening is called a patent foramen ovale.

As shown in FIGS. 4a and 4b, the patent foramen ovale 38 acts like a flap valve, having a right flap 42 and a left flap 44, between the two atria 32 and 34. Normally, higher pressure in the left atrium 34 keeps the flaps closed. However, during certain conditions, such as when there is increased pressure inside the chest around the heart, the flaps may open and blood may travel from the right atrium 32 to the left atrium 34 (see arrows in FIG. 4a). If a clot is present in the right atrium 32 it can enter the left atrium 34 and travel from there to the brain (causing a stroke) or into a coronary artery (causing a heart attack).

Therefore, it is desirable to close the patent foramen ovale 38 permanently. Turning to FIGS. 4a and 4b, the delivery assembly 50 may be percutaneously introduced into a body vessel 40 and directed into, for example, the right atrium 32 and maneuvered adjacent the patent foramen ovale 38. The outer sheath 56 is retracted proximally from the occlusion device 68. The inner catheter 64 may be used to position the occlusion device 68 within the patent foramen ovale 38 such that a small intestine submucosa (SIS) 74 disposed on the occlusion device 68 is positioned between the right and left flaps 42 and 44. As best shown in FIG. 4b, the occlusion device 68 is positioned with the SIS 74 between and in contact with each of the flaps 42 and 44. The proximal portion 70 anchors the device 68 to the flap 42 to secure one end of the device 68 in place. For example, as shown in FIG. 4b, the proximal portion 70 is disposed over the flap 42 to secure one end of the device 68 in place. Likewise, the distal portion 72 anchors the device 68 to the flap 44 to secure the other end of the device 68 in place. For example, as shown in FIG. 4B, the distal portion 72 is disposed over the flap 44, securing the other end of the device 68 in place. In some embodiments additional securing means may also be used including, for example, sutures. As a result, the flaps 42 and 44 of the patent foramen ovale 38 are held closed and in contact with the SIS 74 of the occlusion device 68 and, as described above, body tissue of the atrial septum 36 will quickly differentiate and grow to completely replace the SIS material, thereby permanently closing the patent foramen ovale 38 with tissue grown from the atrial septum 36.

As a person skilled in the art will readily appreciate, the above description is meant as an illustration implementing the principles this invention. This description is not intended to limit the scope or application of this invention in that the invention is susceptible to modification, variation and change, without departing from the spirit of this invention, as defined in the following claims.

We claim:

1. A vascular occlusion device for occluding a body cavity defined by cavity walls, the device comprising:
an elongate flexible member having an axial length and including a proximal portion extending to a distal portion, a radially compressible substance comprising an extracellular matrix and being disposed between the proximal and distal portions, the substance being configured to promote body tissue growth within the body cavity for occluding the body cavity.

2. The device of claim 1 wherein the elongate flexible member is a longitudinally extending coil.

3. The device of claim 1 wherein the proximal and distal portions of the elongate flexible member are curled into loops.

4. The device of claim 3 wherein the loops are curled about axes substantially perpendicular to the axial length of the elongate flexible member.

5. The device of claim 1 wherein the extracellular matrix further comprises small intestine submucosa.

6. The device of claim 5 wherein the small intestine submucosa is compressed for passage through a lumen of a sheath and is expanded when disposed outside of the lumen of the sheath.

7. The device of claim 1 wherein the proximal portion includes a threaded end.

8. The device of claim 1 wherein the elongate flexible member is made of a shape memory material.

9. The device of claim 1 wherein the shape memory material includes alloys of nickel-titanium (Nitinol).

10. A vascular occlusion device for occluding a body cavity defined by cavity walls, the device comprising:
an elongate flexible member including a proximal portion extending to a distal portion and a radially compressible small intestine submucosa being disposed between the proximal and distal portions, the elongate flexible member being a longitudinally extending coil, the proximal and distal portions being configured to couple the elongate flexible member to the cavity walls to position the small intestine submucosa within the body cavity to promote body tissue growth for occluding the body cavity.

11. The device of claim 10 wherein the proximal and distal portions of the elongate flexible member are curled into loops.

12. The device of claim 10 wherein the elongate flexible member is made of a shape memory material.

13. The device of claim 12 wherein the shape memory material includes alloys of nickel-titanium (Nitinol).

14. A vascular occlusion assembly for occluding a body cavity defined by cavity walls, the assembly comprising:
a delivery apparatus including an outer sheath having a proximal end extending to a distal end and defining a lumen therein, an inner catheter being disposed within the lumen and having a proximal segment extending to a distal segment, the outer sheath being configured to translate axially relative to the inner catheter;
an occlusion device disposed within the lumen including an elongate flexible member having a proximal portion extending to a distal portion and an extracellular matrix disposed therebetween, the proximal portion being releasably coupled to the distal segment of the inner catheter, the extracellular matrix being radially compressible for passage through the lumen and configured to promote body tissue growth when disposed within the body cavity; and
the occlusion device being coaxially arranged within the lumen of the outer sheath such that the extracellular matrix is compressed within the lumen, the occlusion device being deployable through the distal end of the outer sheath by means of relative axial movement of the outer sheath, the extracellular matrix being expanded after deployment of the occlusion device.

15. The assembly of claim 14 wherein the extracellular matrix further comprises small intestine submucosa.

16. The assembly of claim 14 wherein the proximal portion further includes a threaded end, the threaded end releasably coupling the proximal portion of the elongate flexible member to the distal segment of the inner catheter.

17. A method of occluding a body cavity having body walls, the method comprising:
providing an occlusion device comprising an elongate flexible member including a proximal portion extending to a distal portion, a radially compressible extracellular matrix being disposed between the proximal and distal portions, the extracellular matrix including small intestine submucosa and being configured to promote body tissue growth within the body cavity for occluding the body cavity;
expanding the extracellular matrix within the body cavity;
positioning the extracellular matrix to promote body tissue growth; and
attaching the occlusion device to the body walls of the body cavity.

18. The method of claim 17 wherein the body cavity further comprises a patent foramen ovale.

19. The method of claim 17 wherein the extracellular matrix further comprises small intestine submucosa.
